(19) United States
(12) Patent Application Publication
(10) Pub. No.: US 2011/0186299 A1
(43) Pub. Date: Aug. 4, 2011

(54) Low-Toxicity Biodegradable Corrosion Inhibitors

(76) Inventors: Tong Eak Pou, Irigny (FR); Jean-Philippe Gillet, Brignais (FR); Christian Gancet, Lons (FR)

(21) Appl. No.: 13/063,783

(22) PCT Filed: Sep. 17, 2009
(86) PCT No.: PCT/FR2009/051745

§ 371 Date: Apr. 1, 2011

(30) Foreign Application Priority Data
Sep. 18, 2008 (FR) 08.56293

(51) Int. Cl. C23F 11/14; C07D 233/24; E21B 43/00

(52) U.S. Cl. 166/310; 548/348.1; 507/243

(57) ABSTRACT

The present invention relates to low-toxicity biodegradable compounds that inhibit metal corrosion, said compounds being salts obtained through the reaction of a carboxylic diacid with an imidazoline derivative. The invention also relates to the use of said corrosion-inhibiting compounds in all types of industries for drilling ore or fossil compounds, such as gas or oil.

The present invention relates to compounds which are inhibitors of the corrosion of metals and which are of low toxicity and biodegradable. The invention also relates to the use of these corrosion-inhibiting compounds in particular in the oil industry and more generally in any type of industry for the drilling of ores or fossil compounds, such as gas or oil.

In oil or gas production, the corrosion of the materials from which the drilling installations, such as platforms, pipelines, valves and other equipment, are constructed is a genuine problem which requires numerous maintenance and repair operations. The corrosion of metals in these industrial categories consequently represents a very significant cost.

The use of corrosion inhibitors is often an advantageous economic solution. However, corrosion inhibitors, in addition to their intrinsic corrosion-inhibiting property, must not have a harmful effect on the environment.

The chemical molecules known today to be good corrosion inhibitors in combating carbon dioxide corrosion (due to CO₂) or hydrogen sulphide corrosion (due to H₂S) are generally imidazolines, amines and derivatives, quaternary ammonium salts and phosphoric esters. However, these molecules suffer from a major disadvantage in that they are harmful to the environment.

It is also known to modify the chemical structure of some organonitrogen molecules in order to render them less toxic, as described, for example, by J. P. Clewlow et al. (U.S. Pat. No. 5,427,999), who have described the reduction in the toxicity of amines or imidazolines by reaction with acrylic acids. According to R. L. Martin et al. (U.S. Pat. No. 5,785,895), the combination between an N-ethoxyimidazoline substituted in the 2 position and a phosphate ester obtained from phosphoric acid and an ethoxylated C₈-C₁₀ alcohol (Alfol 8-10) results in a compound which is of low toxicity.

A. Naraghi et al. (U.S. Pat. No. 6,475,431) teach that the molecules resulting from the reaction between an amidoamine, an unsaturated carboxylic acid (such as acrylic acid) and monochloracetic acid are effective against corrosion in the oil industry and are of low toxicity.

However, a need remains for corrosion-inhibiting compounds which are even more effective and even less toxic and which in particular exhibit a high biodegradability.

The Applicant Company has thus discovered, entirely unexpectedly, that it is possible to increase the biodegradability and reduce the toxicity of a compound of imidazoline type while maintaining good corrosion-inhibiting properties, in particular for the various installations used in the oil and gas industries.

Thus, according to a first aspect, the present invention relates to novel imidazoline carboxylates of following formula (1):

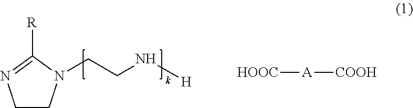

in which:
R represents a saturated or unsaturated and linear or branched, preferably linear, hydrocarbon radical comprising from 7 to 21 carbon atoms, preferably from 11 to 17 carbon atoms,
k represents 1, 2, 3 or 4,
A represents a saturated or unsaturated and linear or branched hydrocarbon chain comprising from 1 to 8 carbon atoms which is optionally substituted with one or more hydroxyl (—OH) and/or carboxyl (—COOH) groups.

The term “unsaturated” used in the definition of the imidazoline carboxylates (or salts) of above formula (1) indicates the presence of one or more unsaturations in the form of double and/or triple bond(s), preferably in the form of double bond(s).

Preference is given to the carboxylates of formula (1) above exhibiting one, preferably several, more preferably all, of the following characteristics, taken in isolation or in combination:
R represents a saturated or unsaturated and linear or branched, preferably linear, hydrocarbon radical comprising from 11 to 17 carbon atoms,
k represents 1 or 2,
A represents a saturated or unsaturated linear hydrocarbon chain comprising from 1 to 4 carbon atoms which is optionally substituted with one or more hydroxyl (—OH) groups; preferably -A- represents —CH₂—CH₂—, —CH═CH—, —CH₂—CH(OH)—, —CH(OH)—CH(OH)— or —(CH₂)₃—.

According to an even more preferred aspect, the imidazoline salts (carboxylates) defined above are N-aminoethyl-2-heptadecenylimidazoline carboxylates. Altogether preferably, the carboxylates according to the invention are chosen from N-aminoethyl-2-heptadecenylimidazoline succinate, maleate, malate, tartrate and glutarate.

The imidazoline salts (1) according to the present invention are advantageously obtained by salification of at least one imidazoline derivative of formula (1a):

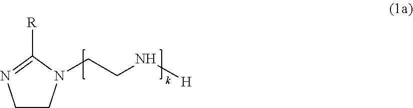

with at least one dicarboxylic acid of formula (1b):

HOOC-A-COOH (1b)

in which formulae (1a) and (1b) R, k and A are as defined above.

The salification reaction can be carried out according to any method commonly used and known to a person skilled in the art. The imidazoline carboxylates of formula (1) can, for example, be easily obtained by bringing at least one imidazoline derivative of formula (1a) into contact with at least one dicarboxylic acid of formula (1b) and then heating the reaction mixture, with stirring.

The reaction temperature can vary within wide limits, according to the nature of the imidazoline derivatives and diacids employed. The reaction solvent can be water, one or more solvents, preferably water-soluble solvents, or also a water/water-soluble solvent(s) mixture.

The imidazoline derivatives of formula (1a) which can be used for the synthesis of the salts of formula (1) are either known, commercially available or easily prepared from known procedures or procedures adapted from known procedures available in the scientific literature, the patent literature, Chemical Abstracts or on the Internet.

According to a preferred embodiment, the imidazoline derivatives (1a) are chosen from alkylimidazolines, preferably from N-aminoethyl-2-undecylimidazoline, N-(aminoethyl)aminoethyl-2-heptadecenylimidazoline and N-aminoethyl-2-heptadecenylimidazoline, and the mixtures of two or more alkylimidazolines. According to an altogether preferred embodiment, the imidazoline derivative of formula (1a) is N-aminoethyl-2-heptadecenylimidazoline (or 2-{2-[(8E)-heptadec-8-enyl]-4,5-dihydro-1H-imidazol-1-yl}ethanamine).

As nonlimiting example, N-aminoethyl-2-heptadecenylimidazoline can advantageously be obtained by a cyclizing reaction between DETA (diethylenetriamine) and oleic acid. The salts of formula (1) according to the present invention thus exhibit the advantage of being able to be prepared, in all or in part, from renewable materials and in particular from fatty acids present in nature, such as the abovementioned oleic acid.

Mention may be made, among dicarboxylic acids of formula (1b) which can be used for the preparation of the salts of formula (1), without implied limitation, of succinic acid (HOOC—CH₂—CH₂—COOH), maleic acid (HOOC—CH═CH—COOH), malic acid (HOOC—CH₂—CH(OH)—COOH), tartaric acid (HOOC—CH(OH)—CH(OH)—COOH), glutaric acid (HOOC—(CH₂)₃—COOH) and other natural, synthetic or artificial dicarboxylic acids, and also the mixtures of two or more of them in all proportions.

According to another embodiment of the present invention, it is also possible to use, as diacid of formula (1b), fatty acid dimers and/or trimers or compositions comprising fatty acid dimers and/or trimers.

Fatty acid dimers or trimers is understood to mean oligomers of 2 or 3 monomers of identical or different monocarboxylic acids, one at least of which is a fatty acid. These oligomers result from the oligomerization of monocarboxylic acids, generally by a condensation reaction on the double bonds, thus resulting in mixtures essentially composed of dimers and trimers. Mention may be made, as preferred examples of fatty acids capable of being oligomerized, of fatty acids comprising unsaturated molecules, for example of oleic type.

Advantageously, the fatty acid oligomers comprise from 12 to 100 carbon atoms and more advantageously still between 24 and 90 carbon atoms. The mixtures of fatty acid oligomers generally comprise a certain level of fatty acid dimers and trimers. The proportion of monomeric fatty acid and of higher fatty acid oligomers (tetramer, pentamer, and the like) is reduced in comparison with the proportion of fatty acid dimers and fatty acid trimers.

Mention may be made, as examples of dimer, of cyclic dimers or linear dimers, including those starting from fatty acids comprising 18 carbon atoms, referred to as C₁₈ acids.

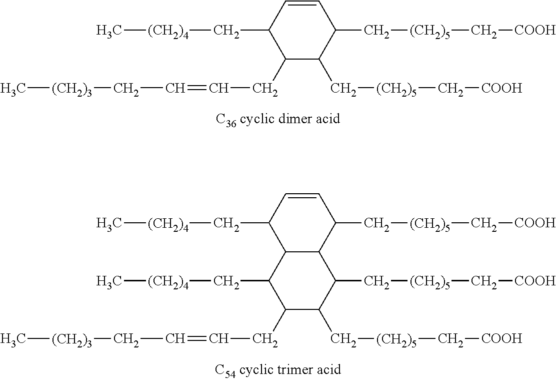

A preferred mixture of fatty acid oligomers comprises dimers, trimers and monomers of C₁₈ fatty acids (linear or cyclic), with a predominant composition of dimers and trimers and a minority of monomers.

A preferred mixture comprises:
from 0.1 to 40% by weight, preferably from 0.1 to 10% by weight, of identical or different fatty acid monomers;
from 0.1 to 99% by weight, preferably from 18 to 98% by weight, of identical or different fatty acid dimers,
from 0.1 to 85% by weight, preferably from 2 to 70% by weight, of identical or different fatty acid trimers.

Mention may be made, as examples of fatty acid dimer/trimer mixtures, of (% by weight):
Pripol® 1017 from Uniqema, mixture of 75-80% of dimers and 18-22% of trimers with of the order of 1-3% of monomeric fatty acid,
Pripol® 1048 from Uniqema, 50/50% mixture of dimers/trimers,
Pripol® 1013 from Uniqema, mixture of 95-98% of dimers and of 2-4% of trimers with a maximum of 0.2% of monomeric fatty acid,
Pripol® 1006 from Uniqema, mixture of 92-98% of dimers and of a maximum of 4% of trimers with a maximum of 0.4% of monomeric fatty acid,
Unidyme® 60 from Arizona Chemicals, mixture of 33% of dimers and of 67% of trimers with less than 1% of monomeric fatty acid,
Unidyme® 40 from Arizona Chemicals, mixture of 65% of dimers and of 35% of trimers with less than 1% of monomeric fatty acid,
Unidyme® 14 from Arizona Chemicals, mixture of 94% of dimers and of less than 5% of trimers and other higher oligomers with of the order of 1% of monomeric fatty acid,
Empol® 1008 from Cognis, mixture of 92% of dimers and 3% of higher oligomers, essentially trimers with of the order of 5% of monomeric fatty acid,
Empol® 1018 from Cognis, mixture of 81% of dimers and of 14% of higher oligomers, including essentially trimers, with of the order of 5% of monomeric fatty acid,
Radiacid® 0980 from Oleon, mixture of the order of 18% of dimers and of 82% of trimers.

As indicated above, the salts of formula (1) according to the present invention can be obtained by reaction between at least one imidazoline derivative of formula (1a) and at least one diacid of formula (1b).

The imidazoline derivative (1a)/diacid (1b) molar ratio is generally between 1/0.1 and 1/5, preferably between 1/0.5 and 1/3, more preferably between 1/1 and 1/2.

According to another subject-matter, the present invention relates to the use of the salts of formula (1) as just defined as corrosion-inhibiting compounds in any type of industry for the drilling of ores or fossil compounds, in particular in the oil and gas industries, and more generally as inhibitors of the corrosion of the pipes in which crude oil or gas is transported.

The salts of the invention can be used, as corrosion inhibitors, alone or in formulation in a water-soluble solvent or a mixture of water-soluble solvents which is(are) preferably of low toxicity and biodegradable. The solvents which can be used are, as nonlimiting examples, water-soluble solvents, such as water, alcohols or glycols, and more specifically water, methanol, ethanol or monoethylene glycol, and the mixtures of two or more of them in all proportions.

Thus, the salts according to the invention can be formulated with water or also with one or more organic solvents or also with water and one or more organic solvents (aqueous/organic formulation).

Altogether advantageously, the components of the said formulation have to form a corrosion-inhibiting formulation compatible with the environment. According to a preferred embodiment, the present invention relates to a formulation comprising from 1% to 90% by weight, preferably from 10% to 30% by weight, of at least one salt of formula (1), from 0% to 20% by weight, preferably from 1% to 10% by weight, of at least one surfactant, advantageously compatible with the environment, and the remainder to 100% by weight of at least one solvent (water or organic or aqueous/organic solvent(s)).

The formulation described above can itself be used as is or can also be diluted, for example immediately before use, in water and/or in one or more solvents, preferably one or more alcohols, such as methanol, ethanol and/or monoethylene glycol.

The surfactants which can be used in the formulation according to the present invention can be of any type among those known to a person skilled in the art, nonionic, ionic or amphoteric.

The compounds according to the present invention, which can be used alone or formulated as indicated above, are highly effective as corrosion inhibitors in any type of industry for the drilling of ores or fossil compounds, such as gas or oil, in particular in the oil and gas industries.

The fluids transported in the pipelines, valves, pumps and other devices are highly corrosive media, due to the presence of a more or less large amount of water saturated with carbon dioxide (CO₂) and/or hydrogen sulphide (H₂S).

The inhibitors of the invention can be used for the treatment by continuous injection, by batch injection or by squeeze injection into the fluids transported in the various pipes, valves, pumps, and the like, of a drilling installation.

According to yet another subject-matter, the present invention relates to the process for preventing or limiting the carbon dioxide corrosion (due to the CO₂ dissolved in the water) and/or the hydrogen sulphide corrosion (due to the H₂S dissolved in the water) of metal parts, in particular of parts made of steel, capable of being damaged by carbon dioxide corrosion and/or by hydrogen sulphide corrosion, the said process comprising bringing the said metal parts into contact with at least one imidazoline carboxylate, as defined above, or at least one formulation, as defined above, comprising imidazoline carboxylate.

The amount of corrosion inhibitor(s) used can vary within wide limits, in particular depending on the type of treatment to be carried out. Generally and without implied limitation, this amount is advantageously between 1 ppm and 10% (weight/volume), with respect to the volume of fluid transported.

More specifically, the amount of corrosion-inhibiting compound can, for example, be between 2 ppm and 50 ppm (weight/volume) for the continuous injection (surface injection), between 100 ppm and 1% (weight/volume) for the batchwise treatment (plugwise to form a film on the pipeline wall) and from 1% to 10% (weight/volume) for the squeeze treatment (injection at the bottom of the oil well as far as the formation).

The present invention also relates to drilling muds, crude oil, gas and others comprising at least one imidazolidine carboxylate as defined above and in particular in an amount of between 1 ppm and 10% (weight/volume), with respect to the volume of fluid transported.

The examples which follow are provided by way of illustration and do not have the purpose of limiting the scope of the present invention defined by the appended claims.

### EXAMPLE 1

### Preparation of the Compounds of the Invention (General Process)

The compounds are synthesized by reaction between a dicarboxylic acid and an imidazoline derivative. By way of example, the imidazoline derivative can be N-aminoethyl-2-heptadecenylimidazoline, itself obtained from oleic acid and diethylenetriamine (DETA) according to conventional processes known to a person skilled in the art.

The reaction can be carried out by direct addition of the solid diacid to the substituted imidazoline but, in particular for reasons related to the viscosity of the medium, it is also possible to use a solution or a suspension of the diacid in ethylene glycol which is run onto the imidazoline.

The diacid/imidazoline derivative molar ratios are between 1/1 and 2/1.

### EXAMPLE 2

### Preparation of N-aminoethyl-2-heptadecenylimidazoline maleate

A suspension of 34.7 g (0.3 mol) of maleic acid in 69 g of ethylene glycol is prepared. This suspension is run on 103 g (0.3 mol) of N-aminoethyl-2-heptadecenylimidazoline (imidazoline A), available from Ceca, and maintained at 40° C. in a reactor with mechanical stirring. Stirring is subsequently continued at this temperature for 2 hours. A homogeneous viscous oil with a solids content of 64.5% is thus obtained.

Similarly, the compounds of Examples 3 to 6 are obtained by varying the nature of the dicarboxylic acid. These compounds are obtained at approximately 50% by weight in monoethylene glycol. The compounds of Examples 3 to 6 are listed in the following Table 1:

TABLE 1

| Example No. | Imidazoline/dicarboxylic acid salt |
|---|---|
| 3 | Imidazoline A/Succinic acid (1/1) |
| 4 | Imidazoline A/Malic acid (1/1) |
| 5 | Imidazoline A/Tartaric acid (1/1) |
| 6 | Imidazoline A/Glutaric acid (1/1) |

### EXAMPLE 7

### Ecotoxicity of the Molecules Studied

### a) Algal Toxicity

The toxicity of the substances for the environment can be measured with regard to various standardized tests. One of the more sensitive consists of the measurement of the toxicity with regard to freshwater algae (Pseudokirchneriella subcapitata). The test is carried out according to the OECD Guideline 201. It consists in evaluating the inhibition of the growth of the algae over a period of time of 72 hours. The characteristic parameter is the EC₅₀, which is the concentration of the substance which brings about inhibition of 50% of the algal growth during the test.

The EC₅₀ values of the products tested are given in Table 2 below:

TABLE 2

| | Algal toxicity |
|---|---|
| Compounds | (EC₅₀ in mg/l) |
| Imidazoline A at 50% by weight in | 0.0047 |
| monoethylene glycol | |
| Example 2 | 1 |
| Example 3 | 0.13 |
| Example 4 | 0.29 |
| Example 5 | 0.14 |

### 2) Biodegradability in Seawater

The biodegradability tests are carried out in a marine environment according to the OECD Guideline 306. The results obtained are given in Table 3 below:

TABLE 3

| | OECD 306 Biodegradability, |
|---|---|
| Compounds | % of biodegradation in 28 days |
| Imidazoline A at 50% by weight in | 57 |
| monoethylene glycol | |
| Example 2 | 66 |
| Example 3 | 62 |
| Example 5 | 59 |

Unexpectedly, the compounds of the invention, which are salts of dicarboxylic acids and of imidazoline derivatives, are markedly less toxic than the unsalified imidazoline alone (factor 30 to 200) and have an entirely comparable, indeed even improved, biodegradability.

### EXAMPLE 8

### Rates of Corrosion in the Absence and in the Presence of the Compounds of the Invention

### 8.1. Carbon Dioxide Corrosion

The rates of carbon dioxide corrosion are measured by the polarization resistance measurement method using a jacketed corrosion cell comprising a three-electrode system (carbon steel test electrode, saturated calomel reference electrode and platinum counterelectrode) under the following conditions:

a) Two-phase corrosive medium:
20% of white spirit
80% of 1 g/l sodium chloride (NaCl) solution

The above mixture (two-phase corrosive medium) is deaerated, by sparging with nitrogen, and then saturated with carbon dioxide (CO₂), by sparging with this gas. The mixture is subsequently introduced into the jacketed corrosion cell described above.

The operating temperature is 80° C. The dosage is 25 ppm (volume/volume) of test compound at 50% by weight on a dry basis in monoethylene glycol with respect to the two-phase medium: 25 microlitres of the test formulation are added to a volume of 1 litre of corrosive medium (20% of white spirit+80% of 1 g/l aqueous sodium chloride solution).

Once everything is in place, the test electrode, the reference electrode and the counterelectrode in the aqueous phase and at 80° C. with magnetic stirring at approximately 100 revolutions per minute, the test formulation is injected into the oil phase (white spirit). The change in the rate of corrosion of the test electrode, in the aqueous phase, is monitored for at least 2 hours, that is to say until stabilization over time is achieved.

Once the rate of corrosion of the control has stabilized (rate of corrosion of the carbon steel without inhibitor, that is to say before the addition of corrosion inhibitor), 25 ppm by volume of test compound are introduced into the oil phase and the rate of corrosion of the test electrode in the aqueous phase is monitored.

The results are given in Table 4.

TABLE 4

| Test compound | Rcor (mm/year) |
|---|---|
| Control | 3.5 |
| Reference (imidazoline A at 50% by | 0.04 |
| weight in monoethylene glycol) | |
| Example 2 | 0.05 |
| Example 3 | 0.07 |
| Example 4 | 0.08 |
| Example 5 | 0.1 |
| Example 6 | 0.08 |

The compounds of the invention (salts obtained by reaction between the dicarboxylic acids and the imidazoline derivatives) show an effectiveness against carbon dioxide corrosion which is entirely comparable to that observed with the use of the imidazoline derivatives alone.

### 8.2. Hydrogen Sulphide Corrosion

The operations described in Example 8.1 of carbon dioxide corrosion are repeated, the two-phase corrosive medium (white spirit and 1 g/l NaCl solution) being this time saturated with hydrogen sulphide (H₂S).

The results obtained are collated in Table 5.

TABLE 5

| Test compound | Rcor (mm/year) |
|---|---|
| Control | 2.5 |
| Reference (imidazoline A at 50% by | 0.04 |
| weight in monoethylene glycol) | |
| Example 2 | 0.02 |
| Example 3 | 0.05 |
| Example 4 | 0.07 |
| Example 5 | 0.08 |
| Example 6 | 0.06 |

The compounds of the invention (salts obtained by reaction between the dicarboxylic acids and the imidazoline derivatives) show an effectiveness against hydrogen sulphide corrosion which is entirely comparable to that observed with the use of the imidazoline derivatives alone.

1. An imidazoline carboxylate of formula (1): 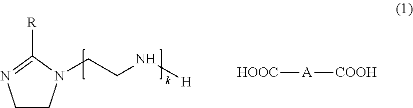
in which:
R represents a saturated or unsaturated and linear or branched hydrocarbon radical comprising from 7 to 21 carbon atoms,
k represents 1, 2, 3 or 4,
A represents a saturated or unsaturated and linear or branched hydrocarbon chain comprising from 1 to 8 carbon atoms which is optionally substituted with one or more hydroxyl (—OH) and/or carboxyl (—COOH) groups.

2. The imidazoline carboxylate according to claim 1, exhibiting one or more of the following characteristics, taken in isolation or in combination:
R represents a saturated or unsaturated and linear or branched hydrocarbon radical comprising from 11 to 17 carbon atoms,
k represents 1 or 2,
A represents a saturated or unsaturated linear hydrocarbon chain comprising from 1 to 4 carbon atoms which is optionally substituted with one or more hydroxyl (—OH) groups.

3. The imidazoline carboxylate according to claim 1, exhibiting the following characteristics, taken in isolation or in combination:
R represents a saturated or unsaturated and linear or branched hydrocarbon radical comprising from 11 to 17 carbon atoms,
k represents 1 or 2,
A represents a saturated or unsaturated linear hydrocarbon chain comprising from 1 to 4 carbon atoms which is optionally substituted with one or more hydroxyl (—OH) groups.

4. The imidazoline carboxylate according to claim 1, wherein it is selected from the group consisting of N-aminoethyl-2-heptadecenylimidazoline succinate, maleate, malate, tartrate and glutarate.

5. A formulation comprising at least one carboxylate according to claim 1 and at least one solvent preferably chosen from water, alcohols, glycols and the mixtures of two or more of them in all proportions.

6. The formulation according to claim 5, comprising:
from 1% to 90% by weight of the at least one carboxylate,
from 0% to 20% by weight of at least one surfactant,
and the remainder to 100% by weight of the at least one solvent.

7. A method of inhibiting corrosion of a drilling installation which comprises contacting the drilling installation with at least one carboxylate according to claim 1.

8. A process for preventing or limiting the carbon dioxide corrosion and/or the hydrogen sulphide corrosion of metal parts capable of being damaged by carbon dioxide corrosion and/or by hydrogen sulphide corrosion, the said process comprising bringing the said metal parts into contact with at least one imidazoline carboxylate according to claim 1.

9. The process according to claim 8, in which the carboxylate is injected continuously, batchwise or by squeeze injection into the fluids transported in the various pipes, valves, or pumps of a drilling installation.

10. The process according to claim 8, in which the amount of imidazoline carboxylate is between 1 ppm and 10% (weight/volume), with respect to the fluid transported, between 2 ppm and 50 ppm (weight/volume) for the continuous injection, or between 100 ppm and 1% (weight/volume) for the batchwise treatment and from 1% to 10% (weight/volume) for the squeeze treatment.

11. A process for drilling mud, crude oil or gas, which comprises using at least one imidazoline carboxylate according to claim 1.

12. The imidazoline carboxylate according claim 1, wherein R represents a saturated or unsaturated linear hydrocarbon radical.

13. The imidazoline carboxylate according claim 1, wherein the hydrocarbon radical comprises 11 to 17 carbon atoms.

14. The imidazoline carboxylate according claim 1, wherein A- represents —CH₂—CH₂—, —CH═CH—, —CH₂—CH(OH)—, —CH(OH)—CH(OH)— or —(CH₂)₃—.

15. The formulation according to claim 6, wherein the carboxylate is present in an amount of 10% to 30% by weight.

16. the formulation according to claim 6, wherein the surfactant is present in an amount of 1% to 10% by weight.
